(19) United States
(12) Patent Application Publication
(10) Pub. No.: US 2026/0151423 A1
(43) Pub. Date: Jun. 4, 2026

(54) Combination Therapy for Brain Cancer

(71) Applicant: Biodexa Ltd., Cardiff South Glamorgan (GB)

(72) Inventors: Katherine Bamsey, Cardiff, South Glamorgan (GB); Valentin Cognet, Cardiff, South Glamorgan (GB); Kelly Conlon, Cardiff, South Glamorgan (GB); Rhian Davies, Cardiff, South Glamorgan (GB); Daniel Palmer, Cardiff, South Glamorgan (GB)

(21) Appl. No.: 19/105,727

(22) PCT Filed: Aug. 26, 2022
(86) PCT No.: PCT/EP2022/073853

§ 371 Date: Feb. 21, 2025

(51) Int. Cl. A61K 31/724; A61K 31/4045; A61K 31/506; A61K 31/52; A61K 31/609; A61P 35/00

(52) U.S. Cl. CPC A61K 31/724; A61K 31/4045; A61K 31/506; A61K 31/52; A61K 31/609; A61P 35/00

(57) ABSTRACT

Combination therapies for use in the treatment of brain tumours, particularly glioma and embryonal brain tumours. Disclosed herein are pharmaceutical compositions comprising a complex of Panobinostat and a cyclodextrin, a second pharmaceutical agent, and a pharmaceutically acceptable excipient, diluent or carrier, in which the second pharmaceutical agent is Niclosamide, Dasatinib or Duvelisib. Associated methods of treatment and use are further disclosed.

### FIELD OF THE INVENTION

The present invention relates to combination therapy for brain tumours, particularly glioma and embryonal brain tumours.

### BACKGROUND

A diffuse intrinsic pontine glioma (DIPG) is a tumour located in the pons of the brain stem. DIPG has a 5-year survival rate of <1%. The median overall survival of children diagnosed with DIPG is approximately 9 months. —the 1- and 2-year survival rates are approximately 30% and less than 10%, respectively. Performing neurosurgery to attempt removal of the tumour is typically infeasible in DIPG patients, due to diffuse invasion of the tumour throughout the brain stem. Conventional treatment therefore involves radiotherapy and chemotherapy. It is known that DIPG tumours frequently exhibit mutations that give rise to altered expression of different tyrosine kinases (MacKay et al., 2017, Cancer Cell, Vol. 32, pp. 520-537). Leptomeningeal disease (LMD) is a rare complication of cancer which occurs as tumour cells invade the cerebrospinal fluid and metastasise to populate the meninges. LMD occurs most frequently in breast cancer, lung cancer, melanoma or skin cancer patients.

Histone deacetylase (HDAC) inhibitors show significant potential for the treatment of various cancers. For example, Panobinostat (Farydak®, LBH-589, 2-(E)-W-hydroxy-3-[4[[[2-(2-methyl-IH-indol-3-yl)ethyl]amino]methyl]phenyl]-2-propenamide) is a non-selective HDAC inhibitor that is used in the treatment of multiple myeloma. HDACs are responsible for regulating the acetylation of a wide variety of cellular proteins. HDAC inhibitors therefore act as effective regulators of multiple biological processes, including DNA replication and repair, chromatin remodelling, transcription of genes, progression of the cell-cycle, protein degradation and cytoskeletal reorganization. For example, the anti-tumour activity of Panobinostat is widely attributed to epigenetic modulation of gene expression and inhibition of protein metabolism. MTX110 refers to a water-soluble formulation of Panobinostat, comprising a Panobinostat-cyclodextrin inclusion complex.

Panobinostat chemotherapy, administered in combination with radiotherapy, has been proposed for use in the treatment of solid tumours (WO2007/050655). WO2021/048412 describes combination therapies comprising Panobinostat, which may find use in the treatment of cholangiocarcinoma. Sohajda, Cyclodextrin News, 2020 reports headline results from the Phase I study of MTX110 monotherapy in patients with DIPG (UCSF study NCT03566199). Truffaux et al., 2015, Neuro-Oncology, Vol. 17(7), pp. 953-964 reports preclinical evaluation of Dasatinib alone and in combination with cabozantinib for the treatment of DIPG. Cutrignelli et al., 2019, Int. J. Mol. Sci., Vol. 20, p. 591, describes a Dasatinib/HP-beta-CD inclusion complex based aqueous formulation as a promising tool for the treatment of paediatric neuromuscular disorders. Broniscer et al., 2013, Clin. Cancer Res., Vol. 19(11), pp. 3050-3058 describes a phase I clinical trial of vandetanib and Dasatinib in children with newly diagnosed DIPG.

Chen et al., 2021, Blood, 138(1) p. 4709 describes the in vitro anti-myeloma effects of Panobinostat alone, and in combination with Duvelisib. Grill et al., 2019, Expert Opinion on Orphan Drugs 7(1) pp. 11-20 is a review article describing new therapeutic developments for the treatment of diffuse intrinsic pontine gliomas (DIPGs). Similarly, Braunstein and colleagues (2017) review the pathology of glial tumours, and their current and future treatments in J. Neurooncol. 134(1) pp. 541-549. WO2017/167837 describes a cyclodextrin-Panobinostat inclusion complex and its use in the treatment of brain cancer, including DIPG.

However, there remains an unmet clinical need within the art for efficacious chemotherapeutic regimens suitable for use in the treatment of brain cancers and metastases infiltrating the brain and neuroaxis. Such chemotherapeutic regimens would find particular clinical utility in patients unsuitable to undertake radiotherapy, for example. The present invention addresses this unmet clinical need, by providing combination therapies as disclosed herein.

### SUMMARY OF THE INVENTION

It is an object of the present invention to provide efficacious chemotherapeutic regimens suitable for use in the treatment of brain cancers.

Accordingly, a first aspect of the invention provides a pharmaceutical composition, comprising: (i) a first pharmaceutical agent comprising a complex of panobinostat and a cyclodextrin; (ii) a second pharmaceutical agent comprising niclosamide, dasatinib and/or duvelisib; and (iii) a pharmaceutically acceptable excipient, diluent or carrier.
In some aspects of the invention, the complex of panobinostat and a cyclodextrin may comprise a β-cyclodextrin, optionally hydroxypropyl-β-cyclodextrin (HPβCD) or sulfobutylether-β-cyclodextrin (SBEβCD). In some aspects, the second pharmaceutical agent may comprise a complex of a cyclodextrin and niclosamide, dasatinib and/or duvelisib. In some aspects, the cyclodextrin of the second pharmaceutical agent may comprise a β-cyclodextrin, optionally a hydroxypropyl-β-cyclodextrin (HPβCD) or a sulfobutylether-β-cyclodextrin (SBEβCD).
In some aspects of the invention, the pharmaceutical composition is an aqueous solution, is a solid or is in lyophilised form. In some aspects, the composition is a lyophilised form suitable for reconstitution in a liquid vehicle. In other aspects, the composition is an aqueous composition.
In some aspects of the invention, the molar ratio of panobinostat to the second pharmaceutical agent is in the range 100:1 to 1:100.

A second aspect of the invention provides use of a pharmaceutical composition of the first aspect of the invention in the preparation of a medicament for use in a method of treatment of a brain cancer in a mammalian subject.

A third aspect of the invention provides a method of treatment of a brain cancer in a mammalian subject, comprising administering to said subject a therapeutically effective amount of a pharmaceutical composition of the first aspect of the invention.

A fourth aspect of the invention provides a method of treatment of a brain cancer in a mammalian subject, comprising administering to said subject a therapeutically effective amount of (i) a complex of panobinostat and a cyclodextrin and (ii) a second pharmaceutical agent comprising niclosamide, dasatinib and/or duvelisib. Optionally, the second pharmaceutical agent may comprise a complex of a cyclodextrin and said niclosamide, dasatinib and/or duvelisib. In some aspects of the invention, the complex of panobinostat and cyclodextrin and the second pharmaceutical agent are administered simultaneously, sequentially or separately to the subject.

A fifth aspect of the invention provides a pharmaceutical composition of the first aspect of the invention for use in a method of treatment of a brain cancer in a mammalian subject.

A sixth aspect of the invention provides a pharmaceutical composition comprising a complex of panobinostat and a cyclodextrin, for use in a method of treatment of a brain cancer in a mammalian subject, in which the method further comprises simultaneously, sequentially or separately administering niclosamide to said subject. The invention also provides a pharmaceutical composition comprising niclosamide, for use in a method of treatment of a brain cancer in a mammalian subject, wherein said method further comprises simultaneously, sequentially or separately administering a complex of panobinostat and a cyclodextrin to said subject. In some aspects, the niclosamide is in the form of a complex of niclosamide and a cyclodextrin, optionally a β-cyclodextrin.

A seventh aspect of the invention provides a pharmaceutical composition comprising a complex of panobinostat and a cyclodextrin, for use in a method of treatment of a brain cancer in a mammalian subject, in which the method further comprises simultaneously, sequentially or separately administering dasatinib to said subject. The invention also provides a pharmaceutical composition comprising dasatinib, for use in a method of treatment of a brain cancer in a mammalian subject, wherein said method further comprises simultaneously, sequentially or separately administering a complex of panobinostat and a cyclodextrin to said subject. In some aspects, the dasatinib is in the form of a complex of dasatinib and a cyclodextrin, optionally a β-cyclodextrin.

An eighth aspect of the invention provides a pharmaceutical composition comprising a complex of panobinostat and a cyclodextrin, for use in a method of treatment of a brain cancer in a mammalian subject, in which the method further comprises simultaneously, sequentially or separately administering duvelisib to said subject. The invention also provides a pharmaceutical composition comprising duvelisib, for use in a method of treatment of a brain cancer in a mammalian subject, wherein said method further comprises simultaneously, sequentially or separately administering a complex of panobinostat and a cyclodextrin to said subject. In some aspects, the duvelisib is in the form of a complex of duvelisib and a cyclodextrin, optionally a β-cyclodextrin.

In some aspects of the second, third, fourth, fifth, sixth, seventh or eighth aspects of the invention, the brain cancer may comprise a leptomeningeal cancer. In some aspects, the brain cancer comprises a glioma or an embryonal tumour. In some aspects, the glioma may comprise a glioma selected from the group consisting of: diffuse intrinsic pontine glioma (DIPG), high grade glioma (HGG), glioblastoma (GBM) and ependymoma. In some aspects, the embryonal tumour comprises medulloblastoma. The glioma may also comprise a glioma selected from the group consisting of: adult-type diffuse gliomas (including glioblastoma IDH-mutant or wildtype), astrocytoma, oligodendroglioma, glioblastoma, angiocentric glioma, polymorphous low-grade neuroepithelial tumour of the young, diffuse low-grade glioma, paediatric-type diffuse high-grade glioma, diffuse midline glioma, diffuse hemispheric glioma, diffuse paediatric-type high-grade glioma (including diffuse midline glioma, H3 K27 altered) and infant-type hemispheric glioma. In some aspects, the brain cancer comprises at least one mutation selected from the group consisting of: histone H3.1K27M, H3.3G34R/V and H3.3K27M. In some aspects of the second, third, fourth, fifth, sixth, seventh or eighth aspects of the invention, the method further comprises analysing a sample obtained from the subject and determining that the brain cancer of said subject comprises at least one mutation selected from the group consisting of: histone H3.1 K27M, H3.3G34R/V and H3.3K27M

In some aspects of the second, third, fourth, fifth, sixth, seventh or eighth aspects of the invention, the method comprises administering the composition to the subject via direct-to-brain infusion. In some aspects, the direct-to-brain infusion may comprise convention enhanced delivery (CED), intra-arterial infusion, intrathecal infusion or fourth ventricle infusion. In some aspects, the infusion is carried out using an Ommaya reservoir. In some aspects, the concentration of panobinostat administered to the subject is between 10 μM and 150 μM. Optionally, the concentration of panobinostat administered to the subject is between 50 μM and 100 μM.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures.

FIG. 1. CellTiter-Glo® (CTG) assay results showing the inhibition induced by MTX110 alone (triangles) or in combination with 0.1 μM Dasatinib-HPβCD (circles) in U118MG cells (A), T98G cells (C), U87MG cells (E) and U251MG cells (G); and showing the inhibition induced by Dasatinib-HPβCD alone (triangles) or with 0.05 μM MTX110 in U118MG cells (B), T98G cells (D), U87MG cells (F) and U251MG cells (H).

FIG. 2. CellTiter-Glo® (CTG) assay results showing the inhibition induced by MTX110 alone (triangles) or with 3 μM Duvelisib-HPβCD (circles) in U118MG cells (A), T98G cells (C), U87MG cells (E) and U251MG cells (G); and showing the inhibition induced by Duvelisib-HPβCD alone (triangles) or with 0.05 μM MTX110 in U118MG cells (B), T98G cells (D), U87MG cells (F) and U251MG cells (H).

FIG. 3. Assessment of the oncolytic activity of Dasatinib, Dasatinib-HPβCD (M323-53-01) and Dasatinib-HPβCD (M323-45-02) in (A) HEP3B (hepatocellular carcinoma) and (B) U87MG (glioblastoma) cells.

FIG. 4. Assessment of the oncolytic activity of 11 different MTX110-containing combination therapies in patient-derived PTX-0166 glioblastoma cells, by DRAQ7® NIR immunofluorescence.

FIG. 5. Assessment of the oncolytic activity of 11 different MTX110-containing combination therapies in patient-derived PTX-0167 glioblastoma cells, by DRAQ7® NIR immunofluorescence.

FIG. 6. Assessment of the oncolytic activity of 11 different MTX110-containing combination therapies in patient-derived PTX-0168 glioblastoma cells, by DRAQ7® NIR immunofluorescence.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Pharmaceutical Agents and Compositions

“Panobinostat”, also known as Farydak®, LBH-589, and 2-(E)-N-hydroxy-3-[4[[[2-(2-methyl-1H-indol-3-yl)ethyl]amino]methyl]phenyl]-2-propenamide is a non-selective HDAC inhibitor having the chemical structure depicted below:

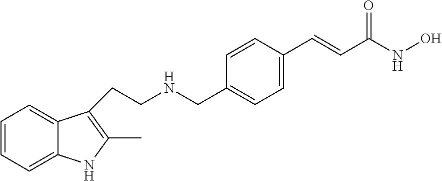 [SMILES: Cc1[nH]c2ccccc2c1CCNCc1ccc(/C=C/C(=O)NO)cc1]

### Panobinostat

As used herein, unless context specifies otherwise (such as when described as “free base Panobinostat”), “Panobinostat” includes salt forms (e.g. Panobinostat citrate, Panobinostat lactate, and the like). Methods for producing hydroxamate derivatives useful as deacetylase inhibitors, including Panobinostat, are detailed in WO2002/022577, the entire content of which is expressly incorporated herein by reference.

“Cyclodextrin” or “CD” is a cyclic oligosaccharide and specifically includes α-cyclodextrins, β-cyclodextrins, and γ-cyclodextrins, and chemically modified forms of these cyclodextrins. The cyclodextrin is typically a pharmaceutically acceptable cyclodextrin. For example, the cyclodextrin may be 2-hydroxypropyl-β-cyclodextrin (HPβCD) or sulfobutylether-β-cyclodextrin (SBEβCD)

“MTX110” or “MTX-110” as used herein refers to a water-soluble form of Panobinostat free base, achieved through complexation with hydroxypropyl-β-cyclodextrin (HPβCD). MTX110 enables convection-enhanced delivery (CED) at potentially chemotherapeutic doses directly to the site of a tumour.

“Convection-enhanced delivery (CED)” is a method of delivering a drug directly to the brain through one or more very small catheters which are surgically placed into or around the brain tumour. The placement of the catheters may be stereotactically directed, e.g. to minimize off-target effects. WO2013/135727, the entire content of which is expressly incorporated herein by reference, describes a method for treatment of glioma by convection enhanced delivery (CED) using a composition of carboplatin in artificial cerebrospinal fluid (CSF). CED typically employs in-line sterilising filters. A number of sterilising filters are not compatible with DMSO (for example, cellulose acetate; cellulose nitrate; polycarbonate; polyether sulfone; Sartobran P; some PVDFs; PVC; Metricet; Nylon; and PES). It is therefore desirable that the compositions of the present invention for CED delivery should be substantially free from certain organic solvents such as DMSO.

As used herein, a “TKI” (tyrosine kinase inhibitor) is any agent that inhibits a tyrosine kinase. A number of TKIs have been approved for use in anti-cancer therapy in humans, or are in development as chemotherapeutic agents. Dasatinib is a tyrosine kinase inhibitor indicated for the treatment of chronic myeloid leukaemia and acute lymphoblastic leukaemia, having the chemical structure depicted below:

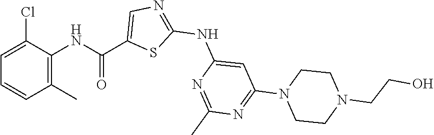 [SMILES: Cc1nc(Nc2ncc(C(=O)Nc3c(C)cccc3Cl)s2)cc(N2CCN(CCO)CC2)n1]

### Dasatinib

Duvelisib is a tyrosine kinase inhibitor indicated for the treatment of chronic lymphocytic leukaemia (CLL), small lymphocytic lymphoma (SLL), and follicular lymphoma, having the chemical structure depicted below:

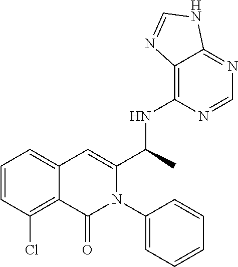 [SMILES: C[C@H](Nc1ncnc2[nH]cnc12)c1cc2cccc(Cl)c2c(=O)n1-c1ccccc1]

### Duvelisib

Dasatinib and Duvelisib, and pharmaceutically acceptable salts thereof, are specifically contemplated for use in accordance with the present disclosure. In some embodiments, Panobinostat is administered to a patient in need thereof simultaneously, sequentially or separately with Dasatinib. In alternative embodiments, Panobinostat is administered to a patient in need thereof simultaneously, sequentially or separately with Duvelisib. In preferred cases, Dasatinib may take the form of a Dasatinib-CD complex, such as Dasatinib-HPβCD. Similarly, in preferred cases, Duvelisib may take the form of a Duvelisib-CD complex, such as Duvelisib-HPβCD.

In addition to Dasatinib and Duvelisib, eleven (11) further pharmaceutical agents were hypothesised to synergistically enhance the oncolytic efficacy of Panobinostat, and were therefore elected for testing as combination therapies. The chemical structures of these pharmaceutical agents are provided in Table 1 below:

TABLE 1
Chemical structures

| Name | Structure |
|---|---|
| Atovaguone | 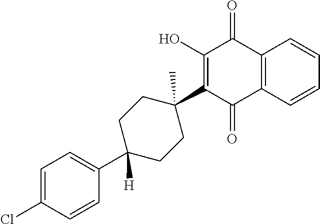 [SMILES: C[C@]1(C2=C(O)C(=O)c3ccccc3C2=O)CC[C@H](c2ccc(Cl)cc2)CC1] |
| Bleomycin | 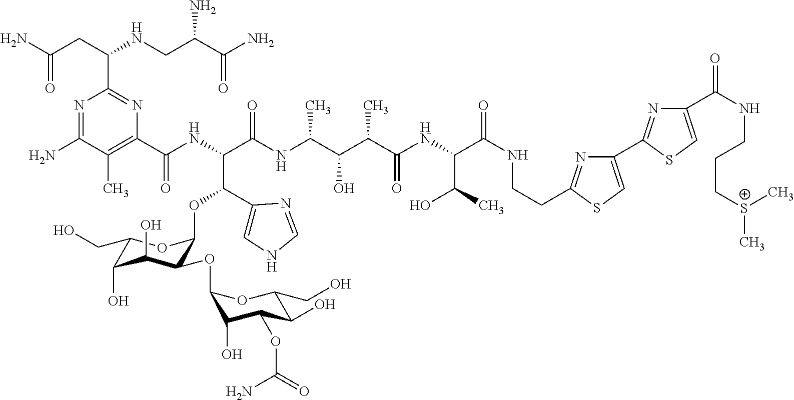 |
| Copanlisib | 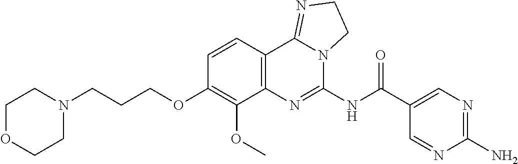 [SMILES: COc1c(OCCCN2CCOCC2)ccc2c1N=C(NC(=O)c1cnc(N)nc1)N1CCN=C21] |
| Bortezomib | 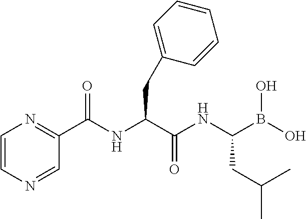 [SMILES: CC(C)C[C@H](NC(=O)[C@H](Cc1ccccc1)NC(=O)c1cnccn1)B(O)O] |
| Etoposide | 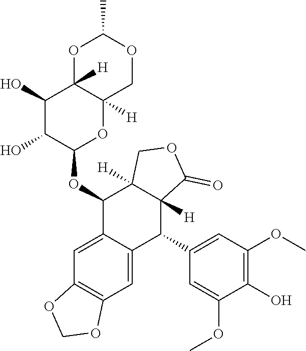 |
| Etomoxir | 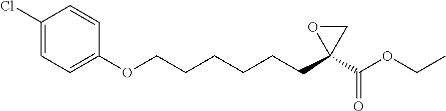 [SMILES: CCOC(=O)[C@@]1(CCCCCCOc2ccc(Cl)cc2)CO1] |
| GSK3787 | 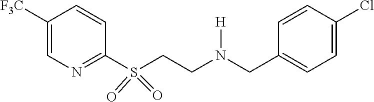 [SMILES: O=S(=O)(CCNCc1ccc(Cl)cc1)c1ccc(C(F)(F)F)cn1] |
| Irinotecan | 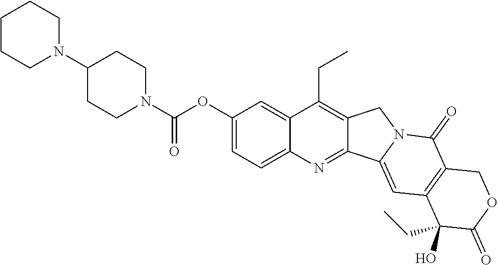 [SMILES: CCc1c2c(nc3ccc(OC(=O)N4CCC(N5CCCCC5)CC4)cc13)-c1cc3c(c(=O)n1C2)COC(=O)[C@]3(O)CC] |
| GSK0660 | 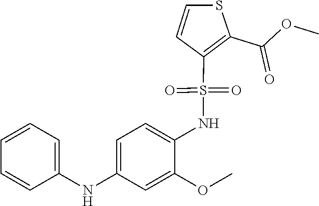 [SMILES: COC(=O)c1sccc1S(=O)(=O)Nc1ccc(Nc2ccccc2)cc1OC] |
| Niclosamide | 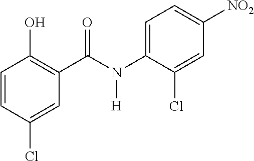 [SMILES: O=C(Nc1ccc([N+](=O)[O-])cc1Cl)c1cc(Cl)ccc1O] |
| Perhexiline maleate | 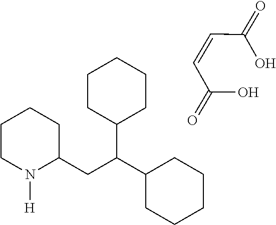 |

Atovaquone, bleomycin, copanlisib, bortezomib, etoposide. etomoxir, GSK3787, irinotecan, GSK0660, niclosamide and perhexiline maleate, and pharmaceutically acceptable salts thereof, are specifically contemplated for use as combination therapies as disclosed herein. In preferred embodiments of the invention, the agents having chemical structures identified in Table 1 may be incorporated into a complex with cyclodextrin (for example, a β-cyclodextrin, such as HPβCD or SBEβCD).

In some embodiments of the invention, panobinostat may be administered to a subject in combination with a second pharmaceutical agent. That is, in some embodiments of the invention, Panobinostat is administered to a subject in need thereof simultaneously, sequentially or separately with atovaquone. In other embodiments, Panobinostat is administered to a subject in need thereof simultaneously, sequentially or separately with bleomycin. In further embodiments, Panobinostat is administered to a subject in need thereof simultaneously, sequentially or separately with copanlisib. Panobinostat may be administered to a subject in need thereof simultaneously, sequentially or separately with bortezomib. Panobinostat may be administered to a subject in need thereof simultaneously, sequentially or separately with etoposide. Panobinostat may be administered to a subject in need thereof simultaneously, sequentially or separately with etomoxir. Panobinostat may be administered to a subject in need thereof simultaneously, sequentially or separately with GSK3787. In further embodiments of the invention, Panobinostat is administered to a subject in need thereof simultaneously, sequentially or separately with irinotecan. Panobinostat may be administered to a subject in need thereof simultaneously, sequentially or separately with GSK0660. Panobinostat may be administered to a subject in need thereof simultaneously, sequentially or separately with perhexiline maleate.

In preferred embodiments of the invention, Panobinostat is administered to a subject in need thereof simultaneously, sequentially or separately with Niclosamide, or a pharmaceutically acceptable salt thereof. In some embodiments, Panobinostat may take the form of a Panobinostat-CD complex, such as MTX110. Similarly, Niclosamide may take the form of a Niclosamide-CD complex, such as Niclosamide-HPβCD or Niclosamide-SBEβCD.

In some aspects of the disclosure, the concentration of Panobinostat administered to the subject is between 10 μM and 150 μM, optionally between 50 μM and 100 μM. Exemplary molar ratios of Panobinostat to the second pharmaceutical agent present in a combination therapies as disclosed herein (e.g., Dasatinib, Duvelisib, and Niclosamide) are from 1:200 to 200:1, preferably from 1:100 to 100:1, more preferably from 1:50 to 50:1, more preferably from 1:10 to 10:1.

A pharmaceutical composition according to the present invention may comprise Panobinostat, a second pharmaceutical agent, and one or more other pharmaceutically acceptable ingredients well known to those skilled in the art, including, but not limited to: pharmaceutically acceptable carriers, diluents, excipients, adjuvants, buffers, pH modifiers, preservatives, anti-oxidants, bacteriostats, stabilisers, suspending agents, solubilisers, surfactants (e.g., wetting agents), colouring agents, and isotonicising solutes (i.e., which render the formulation isotonic with the blood, or other relevant bodily fluid, of the intended recipient). Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 18th edition, Mack Publishing Company, Easton, Pa., 1990; and Handbook of Pharmaceutical Excipients, 2nd edition, 1994. For example, in some embodiments, pharmaceutical compositions as disclosed herein comprise Panobinostat and a pharmaceutically acceptable excipient.

In preferred embodiments of the invention, Panobinostat may take the form of a Panobinostat-CD complex, such as MTX110. Similarly, Dasatinib may take the form of a Dasatinib-CD complex such as Dasatinib-HPβCD; Duvelisib may be a Duvelisib-CD complex, such as Duvelisib-HPβCD; and Niclosamide may take the form of a Niclosamide-CD complex such as Niclosamide-HPβCD.

In some embodiments, the composition is an aqueous solution, is a solid, or is in lyophilised form. For example, the composition may be in lyophilised form suitable for reconstitution in a liquid vehicle. Reconstitution may be effected prior to, e.g. immediately prior to or hours before, administration of the composition to the subject. The reconstitution vehicle may be, e.g., physiological saline, water for injection (WFI), artificial cerebrospinal fluid (aCSF) or other aqueous solution suitable for administration to the brain or cerebrospinal fluid (CSF) of the subject.

In some embodiments the composition is an aqueous composition. It has been found that complexation of Panobinostat and/or a second pharmaceutical agent of the combination therapies disclosed herein with a cyclodextrin (e.g. hydroxypropyl-beta-cyclodextrin (HPβCD) or sulfobutylether-(3-cyclodextrin) (SBEβCD)) allows the active agents to remain stable and soluble in an aqueous solution. In some cases the solution may be stored at a reduced temperature (e.g. 5° C. or lower) prior to use. The aqueous solution may be concentrated relative to the concentration for administration, in which case the composition must be diluted prior to use.

The cyclodextrin may be any cyclodextrin suitable for pharmaceutical use, e.g. an alpha-, beta- or gamma-cyclodextrin, or their chemically modified derivatives. In certain embodiments, the cyclodextrin is a beta-cyclodextrin. For example, the cyclodextrin may comprise sulfobutylether-β-cyclodextrin or 2-hydroxypropyl-β-cyclodextrin.

In some embodiments, the complex of Panobinostat and cyclodextrin, or the second pharmaceutical agent and cyclodextrin may comprise an inclusion complex with a beta-cyclodextrin (e.g. hydroxypropyl-β-cyclodextrin or sulfobutylether-β-cyclodextrin). For example, an inclusion complex of Panobinostat and hydroxypropyl-β-cyclodextrin; an inclusion complex of Dasatinib and hydroxypropyl-β-cyclodextrin, an inclusion complex of Duvelisib and hydroxypropyl-β-cyclodextrin or an inclusion complex of Niclosamide and hydroxypropyl-β-cyclodextrin.

### Administration

In some embodiments, administration to a subject may be carried out by intracavitary delivery (see Mamelak et al. J Clin Oncol., 2006, 24(22), pp. 3644-3650). In some embodiments administration to a subject may be carried out using an Ommaya reservoir (see Blakeley, Curr Neurol Neurosci Rep., 2008, 8(3), pp. 235-241). As used herein “direct to brain” may include CED (for parenchymal infusions, intra-arterial (e.g. basilar artery infusions) and intrathecal or 4th ventricle infusions (e.g. via Ommaya reservoir).

“Artificial cerebrospinal fluid (CSF)” is intended to match the electrolyte concentrations of CSF. Preferably, artificial CSF is prepared from high purity water and analytical grade reagents or can be obtained from medical and commercial suppliers (e.g. South Devon Healthcare NHS Foundation Trust, UK or Tocris Bioscience, Bristol, UK). The final ion concentrations in artificial CSF may be as follows (in mM): Na 150; K 3.0; Ca 1.4; Mg 0.8; P 1.0; Cl 155.

Optionally, each ionic constituent may be at a concentration plus or minus (±) 10%, ±5%, ±2%, ±1% or ±0.5% from the above-listed concentration values. Further optionally, the artificial CSF may comprise glucose and/or one or more proteins at concentrations typically found in human CSF. In preferred cases, the artificial CSF does not comprise glucose or protein.

In some embodiments the Panobinostat-containing combination therapies may be administered in the form of a single pharmaceutical composition, in which the Panobinostat-CD complex (e.g., MTX110) is mixed together with Dasatinib, Duvelisib or Niclosamide. In preferred embodiments, Dasatinib may be a Dasatinib-CD complex, Duvelisib may be a Duvelisib-CD complex and Niclosamide may be a Niclosamide-CD complex.

In some embodiments, the complex of Panobinostat and cyclodextrin (e.g., MTX110) may be administered simultaneously, sequentially or separately to the subject with Dasatinib, Duvelisib or Niclosamide. For example, doses of the Panobinostat-CD complex and Dasatinib, Duvelisib or Niclosamide may be administered within 24 hours of each other. In preferred embodiments, Dasatinib may be a Dasatinib-CD complex, Duvelisib may be a Duvelisib-CD complex and Niclosamide may be a Niclosamide-CD complex.

### Cancers

It is estimated that cancers of the central nervous system (CNS) account for 3% of all annual cancer diagnoses in the UK. Of CNS cancers, brain tumours account for 95% of all diagnoses. As described herein, the term ‘brain cancer’ may refer to one or more cancers selected from the group consisting of: acoustic neuroma; adamantinomatous craniopharyngioma; anaplastic astrocytoma; anaplastic glioma; astrocytoma; atypical teratoid rhabdoid tumour (AT/RT); brainstem glioma; choroid plexus; craniopharyngioma; diffuse astrocytoma; diffuse intrinsic pontine glioma (DIPG); diffuse midline glioma; embryonal tumours; ependymoma; ganglioglioma; germ cell tumour (GCT); glioblastoma multiforme (GBM); glioma; haemangioblastoma; haemangiopericytoma; leptomeningeal metastases, medulloblastoma; meningioma; mixed glioma; neuronal and mixed neuronal-glial tumours; oligodendroglioma; optic nerve/optic pathway glioma; peripheral fibroblastoma; pineal gland tumours; pineal parenchymal tumour with intermediate differentiation (PPTID); pineal region tumours; pituitary tumours; primitive neuroectodermal tumours (PNETs); Schwannoma and solitary fibrous tumours.

In preferred embodiments, the brain cancer comprises a glioma or an embryonal tumour. In such embodiments, the glioma may be selected from the group consisting of: diffuse intrinsic pontine glioma (DIPG), high grade glioma (HGG), glioblastoma (GBM) and ependymoma. The embryonal tumour may comprise medulloblastoma. For the avoidance of doubt, the term ‘brain cancer’ as used herein may also refer to any cancer of the CNS as defined by the WHO 2021 Classification of Tumours of the Central Nervous System (Louis D N, et al. The 2021 WHO Classification of Tumors of the Central Nervous System: a summary. Neuro Oncol. 2021 Aug. 2; 23(8):1231-1251). For example, the term ‘glioma’ may refer to a glioma selected from the group consisting of: adult-type diffuse gliomas (including glioblastoma IDH-mutant or wildtype), astrocytoma, oligodendroglioma, glioblastoma, angiocentric glioma, polymorphous low-grade neuroepithelial tumour of the young, diffuse low-grade glioma, paediatric-type diffuse high-grade glioma, diffuse midline glioma, diffuse hemispheric glioma, diffuse paediatric-type high-grade glioma (including diffuse midline glioma, H3 K27 altered) and infant-type hemispheric glioma.

In preferred embodiments, the brain cancer may comprise a leptomeningeal cancer. For example, leptomeningeal metastases of brain cancers or non-brain cancers (including metastases of solid or haematological cancers). That is, the term brain cancer as used herein expressly encompasses cancer metastases (of any origin) which infiltrate the cerebrospinal fluid (CSF), brain, meninges or the brain-neuroaxis.

In some aspects of the disclosure, the said brain cancer comprises at least one mutation selected from the group consisting of: histone H3.1 K27M, H3.3G34R/V and H3.3K27M. In preferred embodiments, the brain cancer (e.g. a DIPG tumour) may comprise a mutation in histone 3.1 (e.g. H3.1K27M). In some embodiments the subject has previously been tested for the presence of absence of said histone H3.1K27M, H3.3G34R/V and/or H3.3K27M mutations by analysis of a sample obtained from the subject (e.g. a tumour biopsy or a cell-free sample comprising circulating tumour DNA (ctDNA). In some embodiments the method further comprises a step of analysing a sample obtained from the subject and determining that the brain cancer of the subject comprises at least said histone H3.1 K27M, H3.3G34R/V and/or H3.3K27M mutation. For reference, it has been reported that some tumours (e.g., DIPG) bearing the histone H3.3G34R/V or H3.3K27M mutations exhibit overexpression of PDGFRA, c-KIT and KDR (which is also known as VEGFR2) (see MacKay et al., 2017, Cancer Cell, Vol. 32, pp. 520-537).

Accordingly, the present inventors expressly contemplate targeting such tumours with an Panobinostat-containing combination therapy, such as a Panobinostat/Dasatinib; a Panobinostat/Duvelisib or a Panobinostat/Niclosamide combination therapy. In preferred embodiments, Panobinostat may be a Panobinostat-CD complex, such as MTX110. Similarly, Dasatinib may be a Dasatinib-CD complex, Duvelisib may be a Duvelisib-CD complex and Niclosamide may be a Niclosamide-CD complex.

### Therapeutic Uses and Methods of Treatment

Provided herein are methods of treatment of a brain cancer in a mammalian subject. In some embodiments, said methods comprise administering a complex of Panobinostat and a cyclodextrin to a subject simultaneously, sequentially or separately with a second pharmaceutical agent. The second pharmaceutical agent may be Niclosamide (such as a complex of Niclosamide and a cyclodextrin), Dasatinib (such as a complex of Dasatinib and a cyclodextrin) or Duvelisib (such as a complex of Duvelisib and a cyclodextrin). In some embodiments, said methods comprise administering Niclosamide (for example, a complex of Niclosamide and a cyclodextrin) to a subject in need thereof simultaneously, sequentially or separately with Panobinostat (e.g., MTX110). Some methods of the invention may comprise administering Dasatinib (for example, a complex of Dasatinib and a cyclodextrin) to a subject in need thereof simultaneously, sequentially or separately with Panobinostat (e.g., MTX110). Other methods of the invention may comprise administering Duvelisib (for example, a complex of Duvelisib and a cyclodextrin) to a subject in need thereof simultaneously, sequentially or separately with Panobinostat (e.g., MTX110).

In some embodiments, Panobinostat is administered to a subject in need thereof in a single pharmaceutical composition, in which said composition further comprises a second pharmaceutical agent (e.g., Niclosamide, Dasatinib or Duvelisib). Alternatively, a first pharmaceutical composition comprising a complex of Panobinostat and a cyclodextrin may be administered to a subject in need thereof alongside a second pharmaceutical composition comprising the second pharmaceutical agent (such as Dasatinib, Duvelisib or Niclosamide). The second pharmaceutical composition may comprise a complex of the second pharmaceutical agent and a cyclodextrin. Pharmaceutical compositions of the invention may be administered to a subject in need thereof simultaneously, sequentially or separately.

The invention provides pharmaceutical compositions for use in a method of treatment of a brain cancer in a mammalian subject. Suitable pharmaceutical compositions for use in such methods of the invention are disclosed herein.

The invention further provides use of a pharmaceutical composition as disclosed herein in the preparation of a medicament for use in the treatment of a brain cancer in a mammalian subject. Suitable pharmaceutical compositions for use in such aspects of the invention are disclosed herein.

In accordance with the present invention, the mammalian subject may be a human, a companion animal (e.g. a dog or cat), a laboratory animal (e.g. a mouse, rat, rabbit, pig or non-human primate), a domestic or farm animal (e.g. a pig, cow, horse or sheep). Preferably, the subject is a human. In certain embodiments of the invention, for example, in embodiments in which the tumour is a DIPG tumour, the subject may be a human child, e.g. under 10 years of age (such as 1 to 5 years of age).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

The following examples are provided to illustrate the effectiveness of the invention, and are intended to be non-limiting.

### Experiment 1—Panobinostat and Tyrosine Kinase Inhibitor (TKI) Combination Therapies

The present study was designed to assess the therapeutic effect of Panobinostat/TKI combination therapies in vitro. Two tyrosine kinase inhibitors were elected for testing—Dasatinib and Duvelisib.

### Dasatinib

Dasatanib is a tyrosine kinase inhibitor. Activation of RTK has been shown to activate the PI3K-ATK pathway as well as RAS-REF-MEK-MAPK pathways, leading to enhanced cell cycle progression and cell survival. Dasatinib is poorly soluble in water but dissolves in DMSO. Hydroxypropyl-beta-cyclodextrin (HPβCD) is soluble in water but not soluble in DMSO. However, the present inventors have devised a stable Dasatinib-HPβCD formulation suitable for testing.

Four (4) glioblastoma (GBM) cell lines previously shown to be sensitive to Panobinostat with IC₅₀ values of ˜45 nM or below were used for assessment. These cell lines—U251MG, U118MG, T98G and U87MG—are described in detail in Table 2 below.

TABLE 2
Description of GMB cell lines.

| | | Growth | | Medium & |
|---|---|---|---|---|
| Cell line | Cell type | properties | Source | Serum |
| U251MG | Glioblastoma | Adherent | ECACC- | RPMI 1640 + |
| | astrocytoma | | 09063001 | 10% FBS |
| U118MG | Glioblastoma | Adherent | ATCC-HTB-15 | DMEM + 10% |
| | | | | FBS |
| T98G | Glioblastoma | Adherent | ECACC- | EMEM + 10% |
| | | | 92090213 | FBS |
| U87MG | Glioblastoma | Adherent | ATCC-HTB-14 | EMEM + 10% |
| | | | | FBS |

In brief, cells were propagated in flat-bottom Greiner CELLSTAR 96 well plates (product ID # 655090) and maintained in culture conditions at 37° C., in an atmosphere of 5% CO₂ in air. Cells were routinely sub-cultured. Cells in an exponential growth phase were harvested and counted for plating.

Cell viability assays were performed using Promega CellTiter-Glo Luminescent Cell Viability Assay kit (Promega-G7573), following the manufacturer's instructions. In brief, cells were manually counted using a hemocytometer with Trypan blue staining. Cells were adjusted to the following cell densities: U251MG, 3000 cells/well; U118MG, 4000 cells/well; T98G, 3000 cells/well and U87MG, 5000 cells/well. For plating, 90 uL of cell suspension was added to each well of the assay plates. Prior to administration of the test therapeutic compounds, plates were incubated overnight at 37° C., 5% CO₂, 95% air and 100% relative humidity.

To prepare therapeutic compounds for assessment, stock solutions were serially diluted to produce test solutions, as shown in the plate layout (Table 3) below.

TABLE 3
MTX110/Dasatinib-HPβCD plate layout (μM).

| | 1 | 2 | 3 | 4 | 5 | 6 | 7 | 8 | 9 | 10 | 11 | 12 |
|---|---|---|---|---|---|---|---|---|---|---|---|---|
| A | 2500 | 833.3 | 277.8 | 92.59 | 30.86 | 10.29 | 3.429 | 1.143 | 0.381 | Dasatinib- | 250X | Plate |
| | | | | | | | | | | HPβCD | | 1 |
| B | | | | | | | | | 250 | MTX110 | 5000X | |
| C | | | | | | | | | | | | |
| D | 4000 | 1333.3 | 444.4 | 148.1 | 49.38 | 16.46 | 5.487 | 1.829 | 0.610 | MTX110 | 400X | Plate |
| E | | | | | | | | | 100 | Dasatinib- | 1000X | 2 |
| | | | | | | | | | | HPβCD | | |
| F | | | | | | | | | | | | |
| G | | | | | | | | | | | | |
| H | | | | | | | | | | | | |

To produce the 250× solution, (column 11) 8 μL of a concentrated Dasatinib-HPβCD stock solution was diluted in 92 μL assay medium, and serial dilution was completed. 8 μL DMSO was added to blank and control wells to act as a vehicle control. To produce the 5000× solution (column 11), 2 μL of a concentrated MTX110 stock solution was diluted in 498 μL assay medium, and serial dilution was completed. 2 μL DMSO was added to blank and control wells to act as a vehicle control. To produce the 400× (table 11) solution, 5 μL concentrated MTX110 stock solution was diluted in 95 μL assay medium, and serial dilution was completed. 5 μL DMSO was added to blank and control wells to act as a vehicle control. To produce the 1000× (table 11) solution, 6 μL of a concentrated Dasatinib-HPβCD stock solution was diluted in 294 μL assay medium, and serial dilution was completed. 6 μL DMSO was added to blank and control wells to act as a vehicle control.

5 μL of medium containing Dasatinib-HPβCD and 5 μL of medium containing MTX110 were then added into cell-containing wells to initiate the cell viability study. Plates were returned to the incubator and incubated for 3 days. The final DMSO concentration was 0.42% (v/v).

To assess cell death, the CellTiter-Glo buffer and the CellTiter-Glo Substrate were equilibrated to room temperature and mixed to reconstitute the substrate, thereby producing the CellTiter-Glo Reagent. The reagent was mixed by gentle vortexing to obtain a homogenous solution. 50 μL reagent was added to each well (i.e., half the volume of each well) and plates were covered as to be protected from light. Plates were mixed for 2 minutes using an orbital shaker to induce cell lysis and were subsequently incubated on the bench for 10 minutes to allow for stabilization of the luminescent signal. Luminescence was recorded using the 2104 EnVision Multilabel plate reader (PerkinElmer).

The inhibition rate (IR) of tested compounds was determined as per Formula I below. Dose-response curves were plotted and used to evaluate related parameters, including ‘Bottom’, ‘Top’ and IC₅₀ values.

$$I R ( \% ) = ( 1 - ( RLU compound - RLU blank ) \text{} / ( RLU control - RLU blank ) ) * 100 \% \qquad Formula I$$

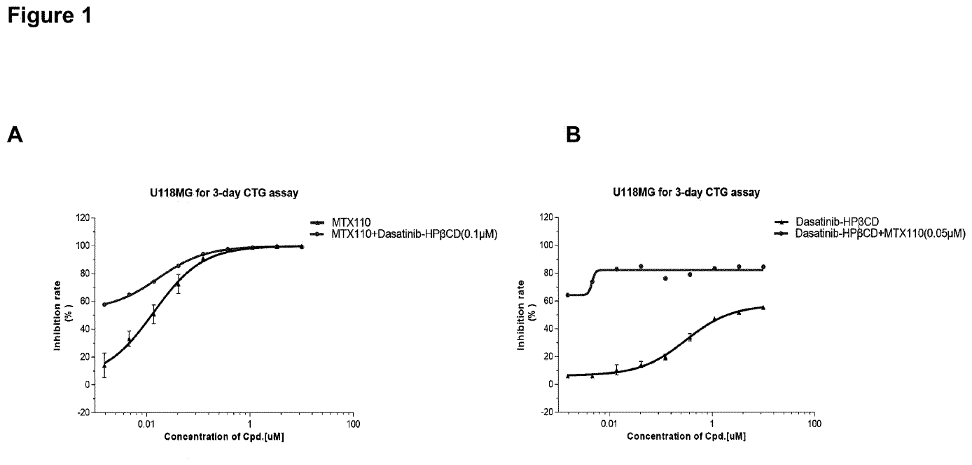

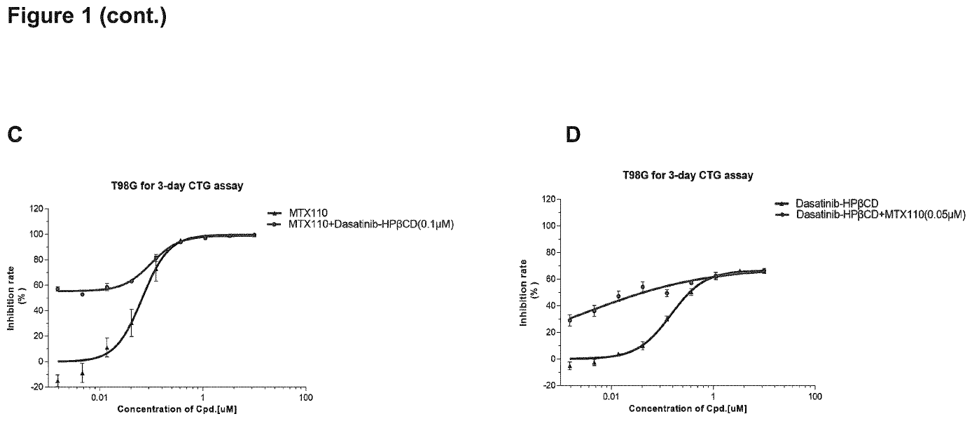

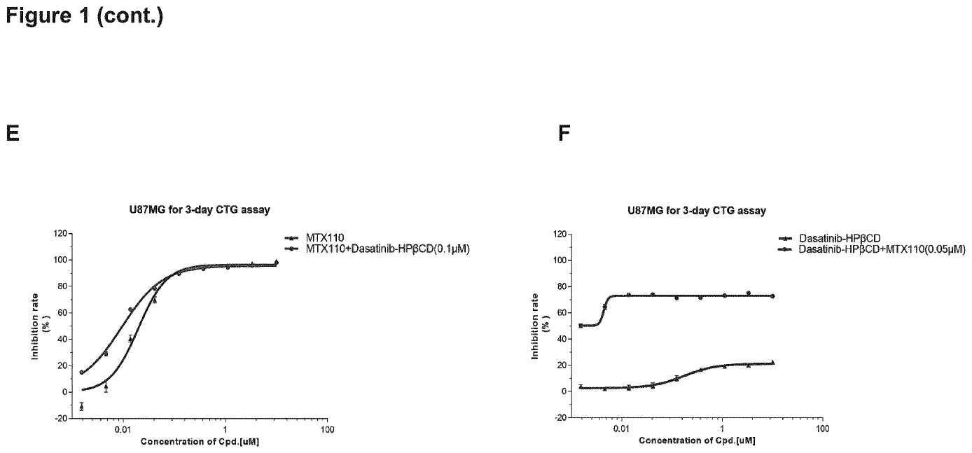

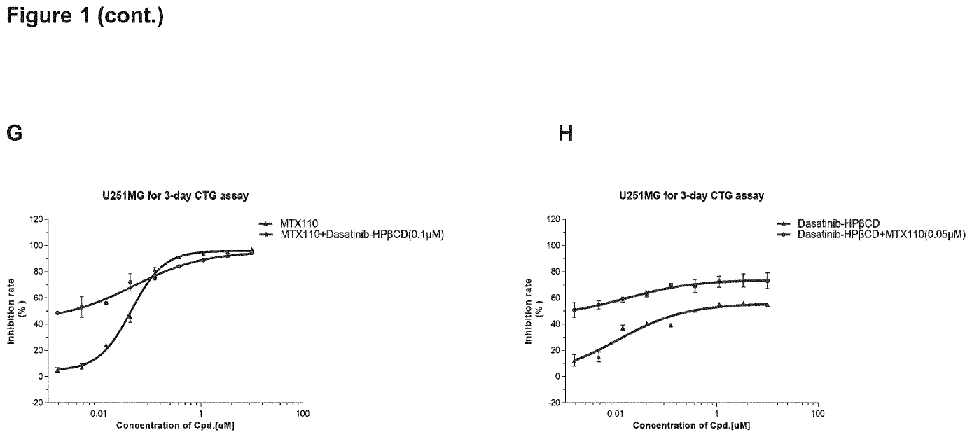

As shown in Table 4 and in FIGS. 1A-H, Dasatinib-HPβCD was found to potentiate the cell-killing activity of Panobinostat-cyclodextrin (i.e. MTX110) at the concentration tested (0.1 μM) and across all four cell lines tested (U118MG, T98G, U87MG and U251MG). Similarly, Panobinostat-cyclodextrin (i.e. MTX110) was found to potentiate the cell-killing activity of Dasatinib-HPβCD against the same cell lines.

TABLE 4
IC₅₀ values for MTX110/Dasatinib-HPβCD combination therapies.

| | | AbsIC₅₀ | ReIC₅₀ | Bottom | Top |
|---|---|---|---|---|---|
| Cell line | Compounds | (μM) | (μM) | (%) | (%) |
| U118MG | MTX110 | 0.012 | 0.014 | 14.07 | 99.71 |
| | MTX110 + Dasatinib- | NA | 0.017 | 57.69 | 99.52 |
| | HPβCD | | | | |
| | Dasatinib-HPβCD | 2.003 | 0.306 | 5.93 | 55.37 |
| | Dasatinib-HPβCD + | NA | 0.005 | 64.25 | 84.83 |
| | MTX110 | | | | |
| T98G | MTX110 | 0.067 | 0.066 | −14.88 | 99.68 |
| | MTX110 + Dasatinib- | NA | 0.099 | 52.86 | 99.82 |
| | HPβCD | | | | |
| | Dasatinib-HPβCD | 0.341 | 0.147 | −5.03 | 66.19 |
| | Dasatinib-HPβCD + | 0.042 | 0.003 | 29.03 | 66.40 |
| | MTX110 | | | | |
| U87MG | MTX110 | 0.021 | 0.020 | −10.81 | 98.98 |
| | MTX110 + Dasatinib- | 0.010 | 0.009 | 15.19 | 98.16 |
| | HPβCD | | | | |
| | Dasatinib-HPβCD | NA | 0.173 | 2.06 | 22.41 |
| | Dasatinib-HPβCD + | NA | 0.004 | 50.28 | 75.17 |
| | MTX110 | | | | |
| U251MG | MTX110 | 0.042 | 0.043 | 5.00 | 97.08 |
| | MTX110 + Dasatinib- | 0.003 | 0.047 | 48.69 | 94.55 |
| | HPβCD | | | | |
| | Dasatinib-HPβCD | 0.305 | 0.011 | 12.40 | 55.86 |
| | Dasatinib-HPβCD + | 0.001 | 0.016 | 50.84 | 73.37 |
| | MTX110 | | | | |

The therapeutic efficacy of MTX110 and Duvelisib-HPβCD-containing combination therapies were investigated utilising experimental protocols equivalent to those outlined above (i.e., as used to assess the efficacy of Dasatinib). Duvelisib is a PI3K inhibitor. PI3K pathway is activated in most GBM patients and leads to pro tumour effects such as enhanced cell survival, enhanced proliferation of cells. The present inventors have devised a stable Duvelisib-HPβCD formulation suitable for testing herein.

To produce a series of test solutions, stock solutions of MTX110 and Duvelisib-HPβCD were serially diluted as shown in the plate layout (Table 5) below.

TABLE 5
MTX110/Duvelisib-HPβCD plate layout (μM).

| | 1 | 2 | 3 | 4 | 5 | 6 | 7 | 8 | 9 | 10 | 11 | 12 |
|---|---|---|---|---|---|---|---|---|---|---|---|---|
| A | 2500 | 833.3 | 277.8 | 92.59 | 30.86 | 10.29 | 3.429 | 1.143 | 0.381 | Duvelisib- | 250X | Plate |
| | | | | | | | | | | HPβCD | | 1 |
| B | | | | | | | | | 250 | MTX110 | 5000X | |
| C | | | | | | | | | | | | |
| D | 4000 | 1333.3 | 444.4 | 148.1 | 49.38 | 16.46 | 5.487 | 1.829 | 0.610 | MTX110 | 400X | Plate |
| E | | | | | | | | | 2500 | Duvelisib- | 833X | 2 |
| | | | | | | | | | | HPβCD | | |
| F | | | | | | | | | | | | |
| G | | | | | | | | | | | | |
| H | | | | | | | | | | | | |

To produce the 250× solution, (column 11) 8 μL of a concentrated Duvelisib-HPβCD stock solution was diluted in 92 μL assay medium, and further serial dilution was completed. 8 μL DMSO was added to blank and control wells to act as a vehicle control. To produce the 5000× solution (column 11), 2 μL of a concentrated MTX110 stock solution was diluted in 498 μL assay medium, and serial dilution was completed. 2 μL DMSO was added to blank and control wells to act as a vehicle control. To produce the 400× (table 11) solution, 5 μL concentrated MTX110 stock solution was diluted in 95 μL assay medium, and serial dilution was completed. 5 μL DMSO was added to blank and control wells to act as a vehicle control. To produce the 833× (table 11) solution, 9 μL of a concentrated Duvelisib-HPβCD stock solution was diluted in 366 μL assay medium, and serial dilution was completed. 9 μL DMSO was added to blank and control wells to act as a vehicle control. 5 μL of medium containing Duvelisib-HPβCD and 5 μL of medium containing MTX110 were then added into cell-containing wells to initiate the cell viability study. Plates were returned to the incubator and incubated for 3 days. The final DMSO concentration was 0.37% (v/v).

The CellTiter-Glo Luminescent Cell Viability Assay was performed as described above, and luminescence was recorded using the 2104 EnVision Multilabel plate reader (PerkinElmer). The inhibition rate (IR) of tested compounds was determined as per Formula I, and dose-response curves were plotted and used to evaluate related parameters (‘Bottom’, ‘Top’ and IC₅₀ values), as shown in Table 6 below.

TABLE 6
IC₅₀ values for MTX110/Duvelisib-HPβCD combination therapies.

| | | AbsIC₅₀ | ReIC₅₀ | Bottom | Top |
|---|---|---|---|---|---|
| Cell line | Compounds | (μM) | (μM) | (%) | (%) |
| U118MG | MTX110 | 0.021 | 0.021 | −0.37 | 99.71 |
| | MTX110 + Duvelisib- | 0.013 | 0.021 | 24.91 | 99.83 |
| | HPβCD | | | | |
| | Duvelisib-HPβCD | NA | 9.541 | −3.60 | 18.16 |
| | Duvelisib-HPβCD + | NA | 0.018 | 69.72 | 99.23 |
| | MTX110 | | | | |
| T98G | MTX110 | 0.066 | 0.065 | −14.22 | 99.83 |
| | MTX110 + Duvelisib- | 0.023 | 0.034 | 21.53 | 99.87 |
| | HPβCD | | | | |
| | Duvelisib-HPβCD | NA | 3.839 | −7.64 | 19.76 |
| | Duvelisib-HPβCD + | NA | 0.029 | 65.11 | 92.62 |
| | MTX110 | | | | |
| U87MG | MTX110 | 0.041 | 0.039 | −21.19 | 98.58 |
| | MTX110 + Duvelisib- | 0.022 | 0.022 | 8.23 | 98.76 |
| | HPβCD | | | | |
| | Duvelisib-HPβCD | NA | 9.172 | −6.69 | 24.38 |
| | Duvelisib-HPβCD + | NA | 0.010 | 62.34 | 95.15 |
| | MTX110 | | | | |
| U251MG | MTX110 | 0.048 | 0.045 | −1.39 | 96.25 |
| | MTX110 + Duvelisib- | 0.036 | 0.036 | 6.80 | 97.01 |
| | HPβCD | | | | |
| | Duvelisib-HPβCD | NA | 1.229 | −1.96 | 3.40 |
| | Duvelisib-HPβCD + | NA | 0.035 | 57.51 | 94.48 |
| | MTX110 | | | | |

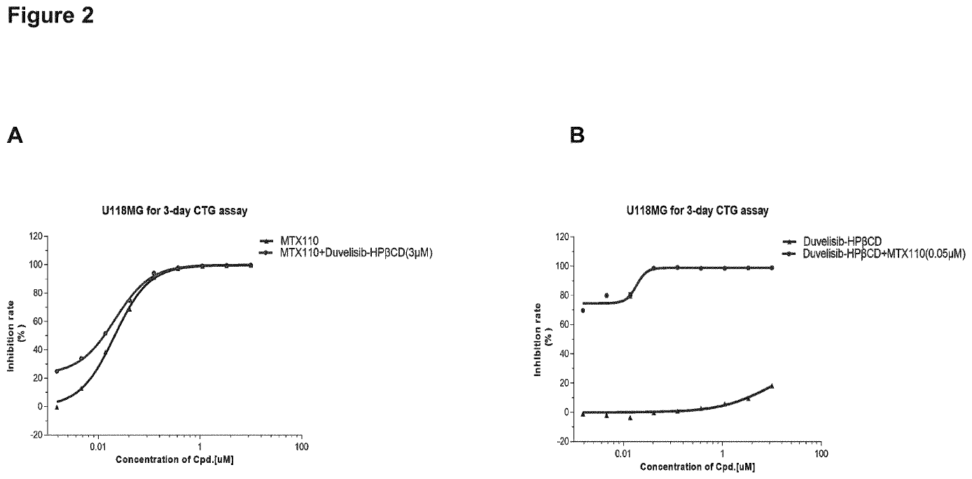

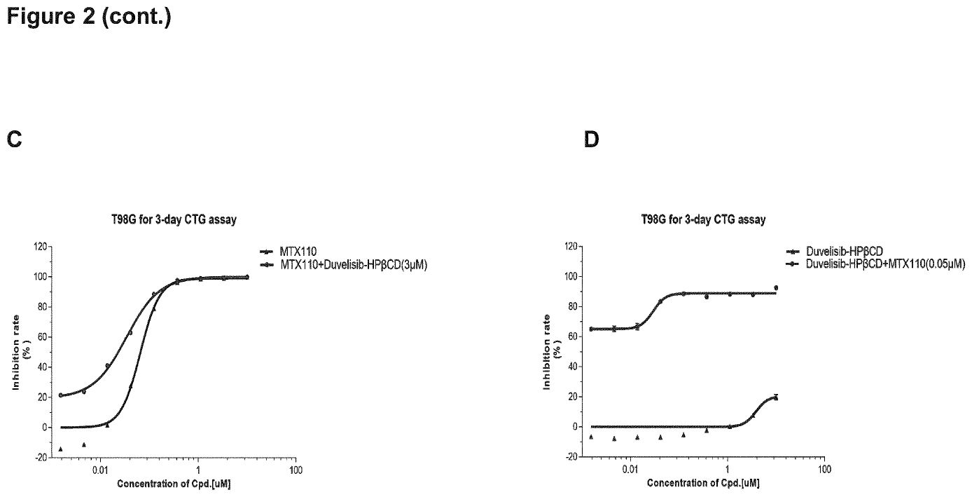

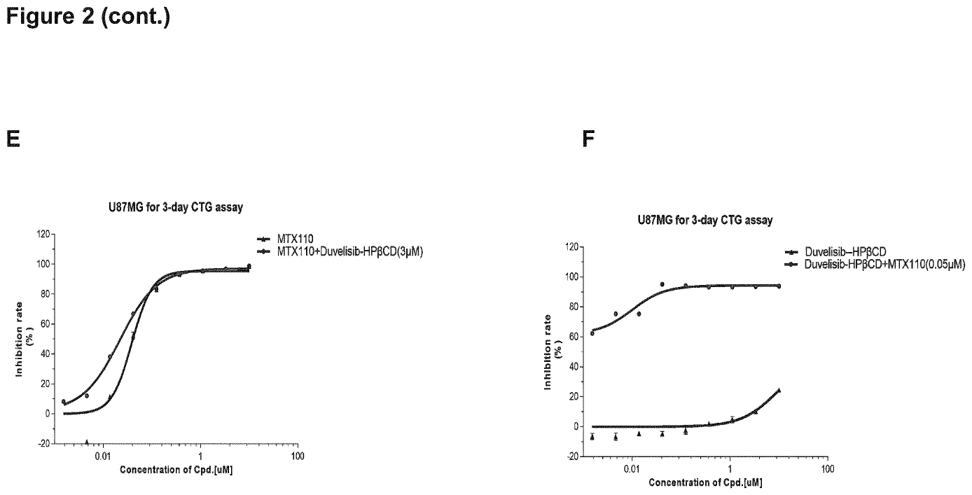

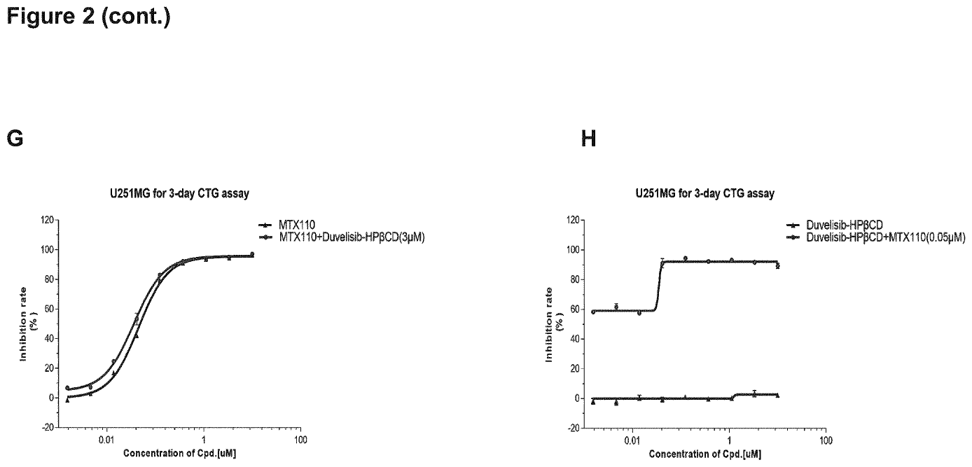

As shown in Table 6 and in FIGS. 2A-H, Duvelisib-HPβCD was found to potentiate the cell-killing activity of Panobinostat-cyclodextrin (i.e. MTX110) at the concentration tested (3 μM) and across all four cell lines tested (U118MG, T98G, U87MG and U251MG). Similarly, Panobinostat-cyclodextrin (i.e. MTX110) was found to potentiate the cell-killing activity of Duvelisib-HPβCD against the same cell lines.

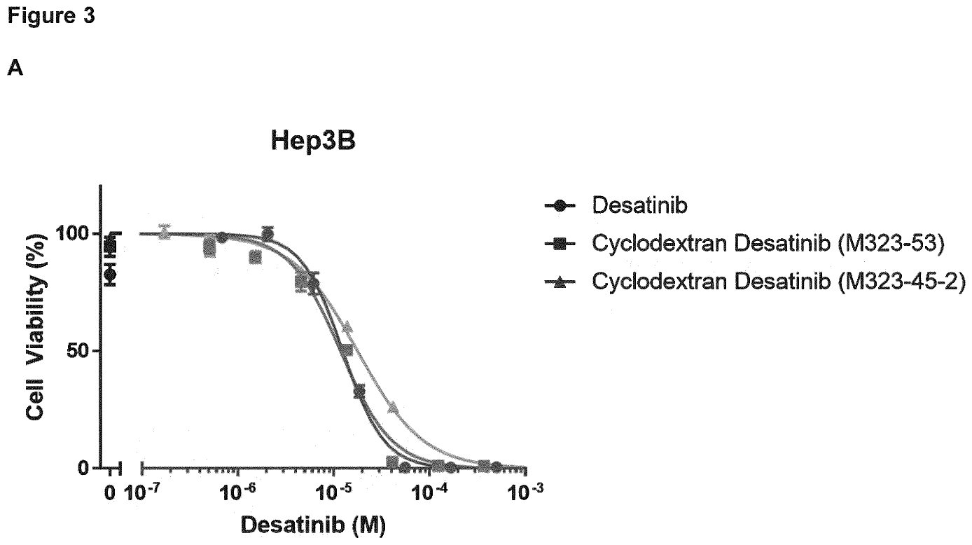

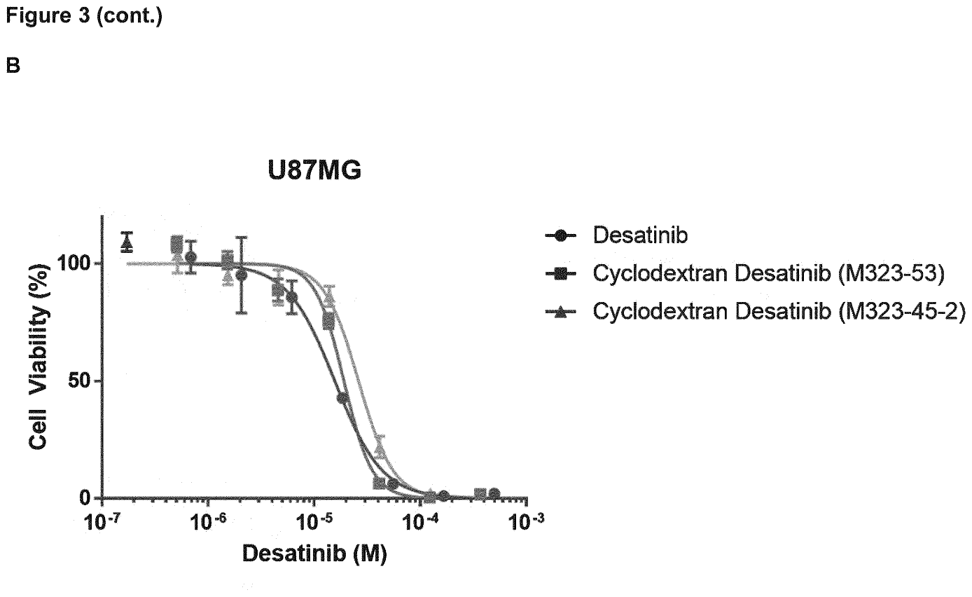

Subsequent analyses were performed to assess the Hill and IC₅₀ values of free Dasatinib and two Dasatinib-HPβCD complexes (termed M323-53-01 and M323-45-02) in Hep3B (hepatocellular carcinoma) and U87MG (glioblastoma) cells. The results are shown in Table 7 and FIG. 3.

TABLE 7
Assessment of Hill and IC₅₀ values of free
Dasatinib and Dasatinib-HPβCD complexes.

| | | | Dasatinib-HPβCD | Dasatinib-HPβCD |
|---|---|---|---|---|
| Cell line | | Dasatinib | (M323-53-01) | (M323-45-02) |
| Hep3B | Hill | 2.0 | 1.6 | 1.3 |
| | IC₅₀ (μM) | 12.5 | 11.8 | 17.8 |
| U87MG | Hill | 1.9 | 3.2 | 2.7 |
| | IC₅₀ (μM) | 15.7 | 19.1 | 26.3 |

Similarly, the MTT of free Dasatinib in the Hep3B (hepatocellular carcinoma) and U87MG (glioblastoma) cell lines was assessed, and compared with that of the Dasatinib-HPβCD M323-53-01 and M323-45-02 complexes. As shown in Table 8, the activity of both Dasatinib-HPβCD complexes was comparable with the free Dasatinib, and was observed to be stable over time.

TABLE 8
MTT of free Dasatinib and Dasatinib-HPβCD complexes

| | U87MG | Hep3B |
|---|---|---|
| Dasatinib | 6.6 μM (95% CI 3.2-13 μM) | 15 μM (95% CI 11-24 μM) |
| M323-53-01 | 14 μM (95% CI 8.2-22 μM) | 14 μM (95% CI 10-20 μM) |
| M323-45-02 | 31 μM (95% CI 15-66 μM) | 29 μM (95% CI 25-35 μM) |

### Experiment 2—MTX110 and Niclosamide Combination Therapies

The present study was designed to assess the therapeutic efficacy of additional APIs (i.e., in addition to Dasatinib and Duvelisib), which it was suspected may synergistically enhance the efficacy of Panobinostat.

A total of eleven (11) APIs were selected for testing. Atovaquone is an antimicrobial medication that is indicated for the prophylaxis and treatment of infection with Pneumocystis species. Bleomycin is a chemotherapeutic agent that is indicated for the treatment of squamous cell carcinoma, metastatic germ-cell cancer and non-Hodgkin's lymphoma. Copanlisib is a chemotherapeutic agent that was originally developed as a medicine to treat marginal zone lymphoma. Bortezomib is a chemotherapeutic agent that is indicated for the treatment of multiple myeloma and mantle cell lymphoma. Etoposide is a chemotherapeutic agent indicated for the treatment of bronchial and testicular cancers, and of lymphomas. Etomoxir is an inhibitor of carnitine palmitoyltransferase-1 (CPT-1), and has been subject to prior clinical development for the treatment of type 2 diabetes mellitus (T2DM) and heart failure. GSK3787 and GSK0660 are experimental. peroxisome proliferator-activated receptor δ (PPARδ) antagonists. Irinotecan is a chemotherapeutic agent indicated for the treatment of metastatic colorectal cancer and metastatic adenocarcinoma of the pancreas. Niclosamide is an anti-helminthic vermifuge/vermicide. Perhexiline maleate is an anti-anginal agent, which targets the voltage-gated potassium channel Kv1.5, HERG, carnitine palmitoyltransferase 1B and carnitine palmitoyltransferase 1A.

Three patient-derived glioblastoma cell lines were utilised for testing: PTX-0166, PTX-0167 and PTX-0168. The PTX-0166 and PTX-0167 lines were taken from patients who had previously received radiotherapy. PTX-0168 was derived from a treatment-naïve patient. PTX-0166 and PTX-0167 both carry a mutation in p53, specifically R196. In contrast, PTX-0168 carries the R175H mutation of p53. Additional details of these cell lines are provided below:

| | | | | |
|---|---|---|---|---|
| PTX-0166 | Glioblastoma | TP53 | R196* | 30Gy in 8 fractions; |
| PTX-0167 | Glioblastoma | TP53 | R196* | 15 fractions 4,005 cGy; |
| PTX-0168 | Glioblastoma | TP53 | R175H | N/A (untreated). |

In brief, cells were cultured following standard laboratory protocols, and maintained in culture conditions at 37° C. in an atmosphere of 5% CO2 in air. Test solutions containing API were prepared from concentrated stock solutions by serial dilution, as shown in Table 9 below.

TABLE 9
API test concentrations (nM)

| | Concentration | Concentration | Concentration |
|---|---|---|---|
| | #1 | #2 | #3 |
| MTX110 | 20 | NA | NA |
| Atovaquone | 300 | 3000 | 10000 |
| Bleomycin | 300 | 100 | 3000 |
| Copanlisib | 10 | 30 | 300 |
| Bortezomib | 0.5 | 1.0 | 5.0 |
| Etoposide | 1500 | 5000 | 15000 |
| Etomoxir | 1000 | 10000 | 50000 |
| GSK3787 | 100 | 1000 | 10000 |
| Irinotecan | 05 | 15 | 50 |
| GSK0660 | 100 | 1000 | 10000 |
| Niclosamide | 100 | 600 | 2000 |
| Perhexiline maleate | 300 | 1000 | 3000 |

Test solutions were administered to cells alone (monotherapy), or in combination with MTX110 at a fixed concentration of 20 nM. Control wells were dosed with an appropriate volume of DMSO, acting as a vehicle control. Plates were incubated for 3 days and percentage (%) cell death was assessed by DRAQ7® immunofluorescence. In brief, DRAQ7® is a membrane-impermeable DNA-binding fluorescent dye that may be used to selectively stain the nuclei of dead/permeabilised cells. Cells were stained following the manufacturers' protocol and using standard laboratory techniques, and imaged under near-infrared (NIR) light. Dead cells were identified and the proportion of cell death in each sample was expressed as a percentage (%) of the overall cell population.

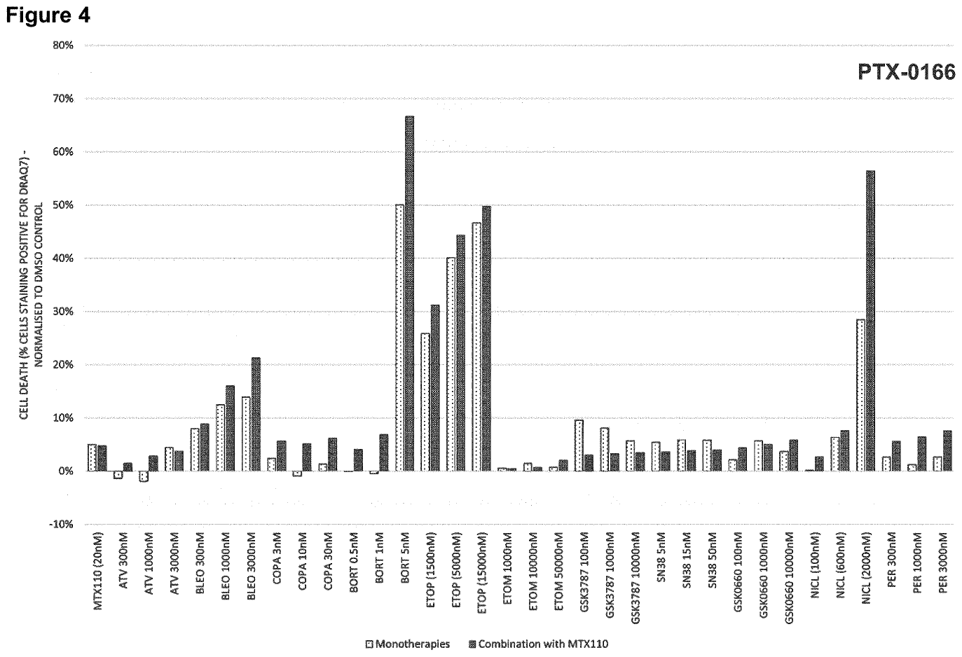

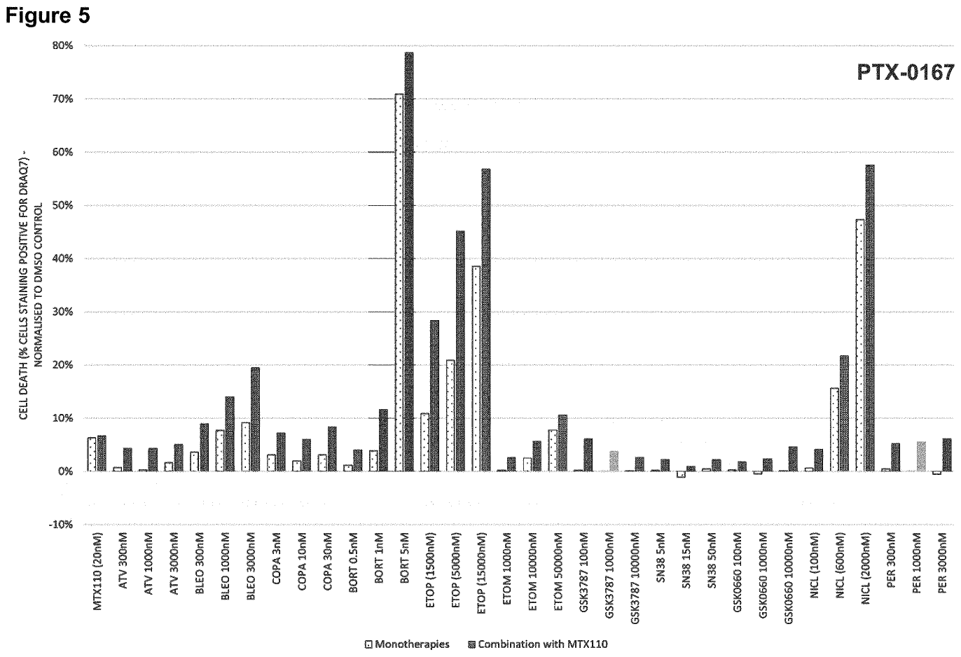

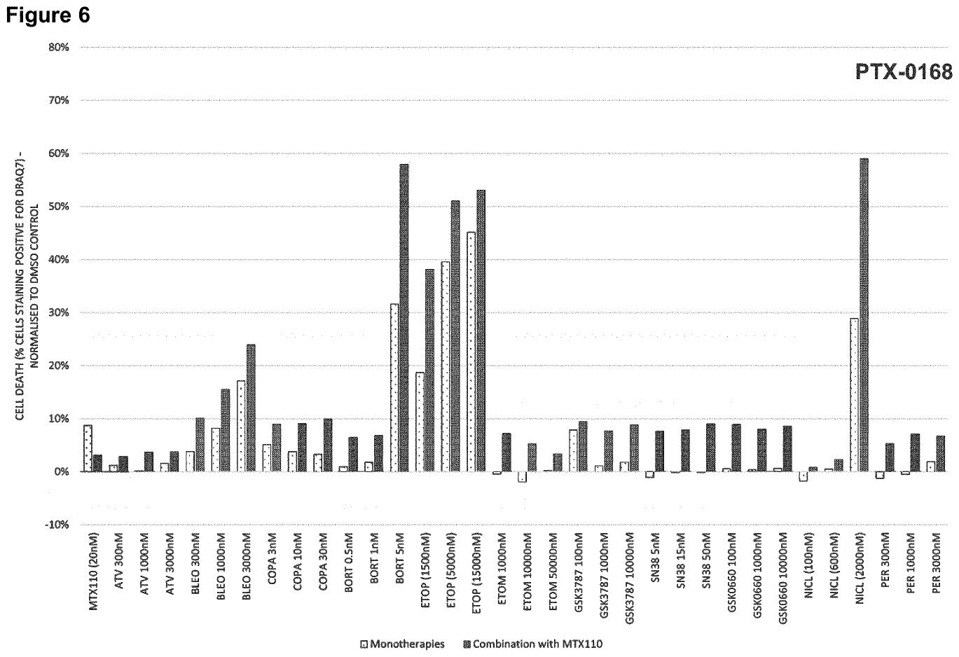

As shown in FIG. 4, FIG. 5 and FIG. 6 MTX110, Etoposide and Niclosamide monotherapies were found to exhibit an oncolytic effect in all cell lines tested. Notably, MTX110 and Niclosamide combination therapy was found to exhibit a substantial synergistic effect. Synergy was identified as the ability of a combination therapy to initiate cell death at a percentage that is higher than would be anticipated following assessment of each constituent therapeutic agent acting alone. Table 10 provides raw data (normalised by subtraction of vehicle control wells) to demonstrate that Niclosamide and MTX110 act synergistically to mediate efficient cell killing—achieving between 56.4 and 59.0% cell death at the highest concentration of Niclosamide tested (2000 nM).

TABLE 10
MTX110/niclosamide combination therapies act synergistically
to mediate efficient cell killing

| PTX-0166 | MTX-110 | Niclosamide | Niclosamide | Niclosamide |
|---|---|---|---|---|
| cells | (20 nM) | (100 nM) | (600 nM) | (2000 nM) |
| Monotherapy | 7.07 | 0.15 | 6.35 | 28.48 |
| Combination | 7.07 | 2.67 | 7.63 | 56.42 |
| therapy | | | | |
| Change | ±0.00 | +2.52 | +1.28 | +27.94 |
| PTX-0167 | MTX-110 | Niclosamide | Niclosamide | Niclosamide |
| cells | (20 nM) | (100 nM) | (600 nM) | (2 μM) |
| Monotherapy | 7.66 | −1.74 | 0.50 | 28.91 |
| Combination | 7.66 | 0.86 | 2.31 | 59.04 |
| therapy | | | | |
| Change | ±0.00 | +2.60 | +1.81 | +30.13 |
| PTX-0168 | MTX-110 | Niclosamide | Niclosamide | Niclosamide |
| cells | (20 nM) | (100 nM) | (600 nM) | (2 μM) |
| Monotherapy | 6.10 | 0.59 | 15.64 | 47.31 |
| Combination | 6.10 | 4.14 | 21.71 | 57.58 |
| therapy | | | | |
| Change | ±0.00 | +3.55 | +6.07 | +10.27 |

### Summary

The data provided in this section act to affirm the efficacy and therapeutic efficacy of the invention. Experiment 1 demonstrates a synergistic interaction between Panobinostat-cyclodextrin and Dasatinib-cyclodextrin; and Panobinostat-cyclodextrin and Duvelisib-cyclodextrin. Experiment 2 demonstrates a synergistic interaction between Panobinostat-cyclodextrin and Niclosamide. The term ‘synergy’ as used herein describes the ability of a combination therapy to initiate higher cell death than could be anticipated from the therapeutic efficacy of each constituent agent acting alone.

In summary, these data successfully demonstrate that Panobinostat-containing combination therapies mediate efficient killing of various glioblastoma cell lines. Panobinostat-containing combination therapies are therefore likely to be valuable clinical tools for use in the treatment of disease.

### REFERENCES

A number of publications are cited above in order to more fully describe and disclose the invention and the state of the art to which the invention pertains. Full citations for these references are provided below. The entirety of each of these references is incorporated herein.
Blakeley, Curr Neurol Neurosci Rep., 2008, 8(3), pp. 235-241
Braunstein et al., 2017, J. Neurooncol. 134(1) pp. 541-549
Broniscer et al., 2013, Clin. Cancer Res., Vol. 19(11), pp. 3050-3058
Chen et al., 2021, Blood, 138(1) p. 4709
Cutrignelli et al., 2019, Int. J. Mol. Sci., Vol. 20, p. 591
Grill et al., 2019, Expert Opinion on Orphan Drugs 7(1) pp. 11-20
Handbook of Pharmaceutical Additives, 2nd Edition (eds. M. Ash and I. Ash), 2001 (Synapse Infor-mation Resources, Inc., Endicott, New York, USA)
Handbook of Pharmaceutical Excipients, 2nd edition, 1994
Louis D N, et al. The 2021 WHO Classification of Tumors of the Central Nervous System: a summary. Neuro Oncol. 2021 Aug. 2; 23(8):1231-1251
MacKay et al., 2017, Cancer Cell, Vol. 32, pp. 520-537
Mamelak et al., J Clin Oncol., 2006, 24(22), pp. 3644-3650
Remington's Pharmaceutical Sciences, 18th edition, MackPublishing Company, Easton, Pa., 1990
Sambrook, J., Russel, D. W. Molecular Cloning, A Laboratory Manual. 3 ed. 2001, Cold Spring Harbor, New York: Cold Spring Harbor Laboratory Press.
Sohajda, Cyclodextrin News, 2020, https://cyclodextrinnews.com/2020/10/20/results-of-hpbcd-enabled-mtx110-phase-i-study/
Truffaux et al. 2015, Neuro-Oncology, Vol. 17(7), pp. 953-964
WO 2002/022577
WO 2007/050655
WO 2013/135727
WO 2017/167837
WO 2021/048412

1. A pharmaceutical composition, comprising:
(i) a first pharmaceutical agent, comprising a complex of panobinostat and a cyclodextrin;
(ii) a second pharmaceutical agent, comprising niclosamide or duvelisib; and
(iii) a pharmaceutically acceptable excipient, diluent or carrier.

2. A method of treatment of a brain cancer in a mammalian subject, comprising administering to said subject a therapeutically effective amount of a pharmaceutical composition comprising:
(i) a first pharmaceutical agent, comprising a complex of panobinostat and a cyclodextrin;
(ii) a second pharmaceutical agent, comprising niclosamide, dasatinib or duvelisib; and
(iii) a pharmaceutically acceptable excipient, diluent or carrier.

3. The pharmaceutical composition of claim 1, wherein the complex of panobinostat and a cyclodextrin comprises a β-cyclodextrin, optionally hydroxypropyl-β-cyclodextrin (HPβCD) or sulfobutylether-β-cyclodextrin (SBEβCD).

4. The pharmaceutical composition of claim 1, wherein the second pharmaceutical agent comprises: a complex of a cyclodextrin and said niclosamide or duvelisib, optionally wherein the cyclodextrin of the second pharmaceutical agent comprises a β-cyclodextrin, optionally hydroxypropyl-β-cyclodextrin (HPβCD) or sulfobutylether-β-cyclodextrin.

5. (canceled)

6. The pharmaceutical composition of claim 1, wherein the composition is an aqueous solution, is a solid or is in lyophilised form, optionally wherein the composition is a lyophilised form suitable for reconstitution in a liquid vehicle.

7. (canceled)

8. The pharmaceutical composition of claim 1, wherein the molar ratio of panobinostat to the second pharmaceutical agent is in the range 100:1 to 1:100.

9. A method of treatment of a brain cancer in a mammalian subject, comprising administering to said subject:
a) a therapeutically effective amount of a pharmaceutical composition comprising a complex of panobinostat and a cyclodextrin, and
b) a therapeutically effective amount of a pharmaceutical composition comprising niclosamide, dasatinib or duvelisib.

10. (canceled)

11. The method of treatment of claim 9, wherein the niclosamide, dasatinib or duvelisib is in the form of a complex of niclosamide and a cyclodextrin, dasatinib and a cyclodextrin, or duvelisib and a cyclodextrin, optionally wherein the cyclodextrin is a β-cyclodextrin.

12. The method of treatment of claim 9, wherein the brain cancer comprises: a leptomeningeal cancer, a glioma or an embryonal tumour.

13. The method of treatment of claim 9, wherein the brain cancer comprises a glioma selected from the group consisting of: diffuse intrinsic pontine glioma (DIPG), high grade glioma (HGG), glioblastoma (GBM) and ependymoma.

14. The method of treatment of claim 9, wherein the brain cancer comprises a glioma selected from the group consisting of: adult-type diffuse gliomas (including glioblastoma IDH-mutant or wildtype), astrocytoma, oligodendroglioma, glioblastoma, angiocentric glioma, polymorphous low-grade neuroepithelial tumour of the young, diffuse low-grade glioma, paediatric-type diffuse high-grade glioma, diffuse midline glioma, diffuse hemispheric glioma, diffuse paediatric-type high-grade glioma (including diffuse midline glioma, H3 K27 altered) and infant-type hemispheric glioma.

15. The method of treatment of claim 9, wherein the brain cancer comprises a medulloblastoma.

16. The method of treatment of claim 9, wherein the brain cancer comprises at least one mutation selected from the group consisting of: histone H3.1K27M, H3.3G34R/V and H3.3K27M, optionally
wherein the method further comprises analysing a sample obtained from the subject and determining that the brain cancer of said subject comprises at least one mutation selected from the group consisting of: histone H3.1K27M, H3.3G34R/V and H3.3K27M.

17. (canceled)

18. The method of treatment of claim 9, wherein the method comprises administering the composition to the subject via direct-to-brain infusion, optionally
wherein the direct-to-brain infusion comprises convention enhanced delivery (CED), intra-arterial infusion, intrathecal infusion or fourth ventricle infusion; and/or
wherein the infusion is carried out using an Ommaya reservoir.

19.-20. (canceled)

21. The method of treatment of claim 9, wherein the concentration of panobinostat administered to the subject is between 10 μM and 150 μM, optionally between 50 μM and 100 μM.

22. The method of treatment of claim 9, wherein the brain cancer is a leptomeningeal cancer.

23. The method of treatment of claim 2, wherein the complex of panobinostat and a cyclodextrin comprises a β-cyclodextrin, optionally hydroxypropyl-β-cyclodextrin (HPβCD) or sulfobutylether-β-cyclodextrin (SBEβCD).

24. The method of treatment of claim 2, wherein the second pharmaceutical agent comprises: a complex of a cyclodextrin and said niclosamide or duvelisib, optionally wherein the cyclodextrin of the second pharmaceutical agent comprises a β-cyclodextrin, optionally hydroxypropyl-β-cyclodextrin (HPβCD) or sulfobutylether-β-cyclodextrin.

25. The method of treatment of claim 2, wherein the composition is an aqueous solution, is a solid or is in lyophilised form, optionally wherein the composition is a lyophilised form suitable for reconstitution in a liquid vehicle.

26. The method of treatment of claim 2, wherein the molar ratio of panobinostat to the second pharmaceutical agent is in the range 100:1 to 1:100.
